SEQUENCE LISTING (4 sequences)

SEQ ID NO: 1 - protein, 14 aa, Artificial Sequence
  synthesized peptide
SGSGSGSGSGSGSG

SEQ ID NO: 2 - protein, 14 aa, Artificial Sequence
  synthesized peptide
SGSGSGSGSGXGXG

SEQ ID NO: 3 - protein, 14 aa, Artifical Sequence
  SITE 5,7,9,11,13: synthesized peptide
SGSGXGXGXGXGXG

SEQ ID NO: 4 - protein, 14 aa, Artifical Sequence
  SITE 1,3,5,7,9,11,13: synthesized peptide
XGXGXGXGXGXGXG

(19) United States
(12) Patent Application Publication
(10) Pub. No.: US 2005/0065068 A1
(43) Pub. Date: Mar. 24, 2005

(54) Methods and compositions for reducing serum phosphate levels

(76) Inventors: Yoshinari Kumagai, Emeryville, CA (US); Akira Otaka, Kuzuha (JP)

Correspondence Address: BOZICEVIC, FIELD & FRANCIS LLP, 1900 UNIVERSITY AVE, SUITE 200, EAST PALO ALTO, CA, 94303, US

(21) Appl. No.: 10/918,082

(22) Filed: Aug. 12, 2004

Related U.S. Application Data
(63) Continuation of application No. 09/642,609, filed on Aug. 17, 2000.
(60) Provisional application No. 60/152,046, filed on Sep. 2, 1999.

(51) Int. Cl. A61K 38/16

(52) U.S. Cl. 514/7

(57) ABSTRACT

Peptidic compounds which significantly reduce serum phosphate levels and which, in addition, reduce bone loss or weakening are provided by the invention. Also provided is a method for treating or preventing any condition associated with elevated serum phosphate levels by administering the peptidic compounds by oral or injectable means.

### FIELD OF THE INVENTION

This invention relates to methods for treating hyperphosphatemia. Particularly, the invention relates to methods and compositions for reducing serum phosphate levels.

### BACKGROUND OF THE INVENTION

Phosphorus is a mineral that plays an integral role in several cellular processes in the body. Phosphorus as an element (P) is extremely unstable when surrounded by oxygen present in the atmosphere or in the environment of normal biological conditions. Because it can be readily oxidized under those conditions, it usually exists in a stable oxidated form of phosphate (PO₄). Phosphate is an essential component of genes (i.e. RNA and DNA polynucleotides) and cell membranes (i.e. in the forms of phosphorylated lipids and sugars). It also plays a key role in energy metabolism and signal transduction in cells as a part of purine phosphate derivatives (ATP, ADP, AMP, GTP, etc.) and phosphorylated sugars, lipids, and proteins. Due to its importance and abundance, control of phosphate level in the body is critical in maintaining proper cellular functions and systemic homeostasis.

Total body phosphorus content is approximately 700 grams, which is equivalent to approximately 2,200 grams as phosphate. About 80% of phosphate in the body is contained in the bones as hydroxyapatite (a complex salt consisting of calcium, phosphate, and hydroxide), and the remainder is located in the soft tissue and extracellular fluid. Of the approximately 3.1-5.6 grams of phosphate ingested each day, most (approximately 70%) is absorbed in the intestine. Upon entering the extracellular fluid, the phosphate is stored in the bone for future mobilization, and/or maintained in the serum. The kidney filters serum inorganic phosphate and approximately 80% of the filtered load is reabsorbed, primarily in the proximal tubule. Thus, the kidney plays a crucial role in maintaining phosphate homeostasis.

When the function of the kidney ceases (i.e. dramatically reduced glomerular filtration rate), the ability of the kidney to excrete phosphate is diminished. Thus, in patients with chronic renal failure, phosphate is retained and hyperphosphatemia ensues. Hyperphosphatemia is one of the major factors in the progression of secondary hyperparathyroidism in chronic renal failure (Felsenfeld, et al. (1999) J. Am. Soc. Nephrol 10(4):878-890; Slatopolsky, et al. (1973) Kidney Int. 4(2):141-145). The excessive secretion of PTH in secondary hyperparathyroidism subsequently leads to the bone disease referred to as renal osteodystrophy, which results in abnormal weakness of the skeleton, due to release of calcium and phosphate from the bone into the body fluid. Another severe complication of high serum phosphate in renal failure patients is soft tissue and vascular calcification. The reduction of serum phosphate level in renal failure patients, then, is a crucial aspect in the management of this patient group.

Chronic renal failure patients are treated with calcitriol (1,25(OH)2D3) to limit the progression of secondary hyperparathyroidism. One of the drawbacks of calcitriol treatment, however, is that calcitriol contributes to phosphate retention. Calcitriol promotes phosphate mobilization from the bone, dietary phosphate absorption from the intestine, and phosphate reabsorption from the kidney (Reichel, et al. (1989) N. Engl. J. Med.320:980-981.

Clinicians attempt to manage hyperphosphatemia in chronic renal failure patients by utilizing dialysis to remove phosphate. Unfortunately, dialysis is relatively inefficient at attaining this goal (Delmez, et al. (1992) Am. J. Kidney 19(4):303-317). Nephrologists also seek to limit a patient's intestinal phosphate absorption by restricting the amount of dietary phosphate ingested and administering tablets that form insoluble phosphate complexes in the gut (i.e.phosphate binders), The major downfall of these approaches is poor patient compliance. A phosphate-restricted diet limits a patient's consumption of protein-rich foods (meat and dairy products), grain breads, and cereals. Patients have difficulty maintaining such a diet because of its bland nature. The issue of poor patient compliance is compounded when considering that these patients also have to consume large quantities of phosphate binders every meal, often without supervision.

Patient compliance is not the only problem concerning the use of phosphate binders. The side effect profile is a concern especially when considering the use of large oral doses of metal salts as phosphate binders with meals. Aluminum-containing salts were, for years, the most frequently used phosphate binder in chronic renal failure patients. Aluminum forms a stable salt with phosphate and is readily deposited into the skeleton. Therefore, aluminum-containing salts were useful in recruiting body fluid phosphate and reabsorbing them in bones, and subsequently reducing body fluid phosphate level. However, aluminum was found to be quite toxic. Accumulation of aluminum in the bone in these patients resulted in osteomalacia and fractures (Faugere, et al., (1986) J. Lab. Clin. Med. 107(6):481-487; Andress, et al., (1987) J. Clin. Endocrinol. Metab. 65(1):11-16; Ward, et al., (1978) Lancet 1(8069):841-845). Bone weakening due to aluminum worsened the secondary bone disease caused by renal failure (i.e. renal osteodystropny). In addition, data implicate a relation between aluminum and dementia in dialysis patients.

Calcium-containing salts (e.g. calcium carbonate, calcium acetate) have also been utilized to reduce serum phosphate levels. Unfortunately, this approach is limited because patients can develop hypercalcemia that may lead to life-threatening ectopic calcification (Emmett, et al., (1991) Am. J. Kidney 17(5):544-550: Slatopolsky, et al., (1986) Semin. Nephrol. 6(4 Supp 1):35-41). In addition, hypercalcemia can be exacerbated when these salts are utilized in combination with calcitriol (Andress, et al., (1989) N. Engl. J. Med. 321(5):274-279). Other complications of calcium-containing salts, particularly calcium carbonate, include gastrointestinal side effects such as constipation and dyspepsia. To a lesser extent, magnesium-containing salts have been used to bind phosphate; however, these salts have been shown to induce high serum magnesium concentrations and diarrhea. Lanthanum-containing and iron-containing salts are also being considered as phosphate binders, but less information regarding these approaches is known. These approaches again seem to be limited by patient compliance (i.e. ingesting large oral doses with every meal) and potential gastrointestinal side effects.

Another phosphate binding agent used in chronic renal failure patients is poly(allylamine-co-N,N′-diallyl-1,3-diamino-2-hydroxypropane) hydrochloride (sevelamer hydrochloride). This nonmetallic agent is advantageous in that the potential for metal induced toxicity is diminished. However, the administration of large oral doses of sevelamer hydrochloride with each meal does not eliminate the concern of patient compliance. Furthermore, gastrointestinal adverse events are common, and long-term studies in large patient groups are required to determine the safety of sevelamer hydrochloride. Similar poly(diallylamine)-based phosphate binders have been disclosed. International Patent Publication WO 99/22743.

Another concern of the phosphate binders is that their collective mode of action may perturb the incorporation of phosphate into the bone. Each of the phosphate binder's function by forming insoluble phosphate complexes in the intestine that are subsequently removed from the body in the feces. An ideal agent for the treatment of hyperphosphatemia should not only lower serum phosphate, but enhance the delivery of excess phosphate to the bone. This is especially critical when considering the bone weakening seen in chronic renal failure patients.

Therefore, there is a significant need to improve upon current hyperphosphatemia treatments. Ultimately, a novel therapeutic approach should not only reduce serum phosphorus levels, but should also facilitate the transfer of phosphate into the bone and promote bone strength. In addition, such an approach should facilitate patient compliance (i.e. a significantly lower oral dosage that does not need to be taken as frequently or with meals) and reduce toxicity (e.g. hypercalcemia, gastrointestinal effects).

### SUMMARY OF THE INVENTION

The present invention provides peptidic compounds and pharmaceutically effective compositions comprising the peptidic compounds. The peptidic compounds of the invention comprise one or more moieties that are phosphorylated, and/or that are capable of being phosphorylated in vitro or in vivo, particularly by physiologic enzymes. In some embodiments, the peptidic compounds comprise repeating (Ser-X) units, wherein X is any amino acid, and which may optionally comprise phosphoserine. In some of these embodiments, the peptidic compounds comprise repeating (Ser-Gly) units, which may optionally comprise phosphoserine. These compounds and compositions, when administered in an effective amount to an individual, are useful in reducing serum phosphate levels in the individual.

The peptidic compounds and compositions comprising the compounds are useful for treating a disease or condition related to hyperphosphatemia. Accordingly, in one aspect, the present invention provides methods of reducing serum phosphate levels in an individual. The methods involve administering to an individual in need thereof a therapeutically effective amount of a composition comprising a peptidic compound of the invention, and preferably repeating the administration over a period of time, thereby reducing serum phosphate levels in the individual.

The peptidic compounds and compositions of the invention are effective in reducing serum phosphate levels in an individual, and provide an additional advantage in that they improve skeletal strength by promoting the incorporation of phosphate into the bone. The treatment methodology is based on the discovery that oral administration of certain series of peptides reduce serum phosphate level, increase bone phosphorus content, and significantly improve bone strength in an in vivo osteoporosis model.

These and other objects, advantages, and features of the invention will become apparent to those persons skilled in the art upon reading the details of the peptidic compounds, compositions, and treatment methods as more fully described below.

### BRIEF DESCRIPTION OF THE DRAWINGS

FIG. 1 shows the average serum phosphate level of all animals in each group of an osteoporosis model as measured on Day 84.

FIG. 2 shows the average serum calcium level of all animals in each group of an osteoporosis model.

FIG. 3 shows the average phosphorus content in the femur of all animals in each group as measured on Day 84.

FIG. 4 shows the average initial bone stiffness in the three point bending mechanical strength test of the femur of all animals in each group as measured on Day 84.

FIG. 5 shows the average maximum load in the three point bending mechanical strength test of the femur of all animals in each group as measured on Day 84.

FIG. 6 shows the average energy at maximum in the three point bending mechanical strength test of the femur of all animals in each group as measured on Day 84.

### MODES OF CARRYING OUT THE INVENTION

Before the present compounds and methods of treatment are described, it is to be understood that this invention is not limited to the particular compounds, methodology or formulations described, as such compounds methods and formulations may, of course, vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example,
reference to “a formulation” includes mixtures of different formulations, reference to “a compound” includes one or more compounds, and reference to “the method of treatment” includes reference to equivalent steps and methods known to those skilled in the art, and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated by reference to describe and disclose specific information for which the reference was cited and with which the reference is connected.

The publications discussed herein are provided solely for their disclosure prior to the filing date of the present application. Nothing herein is to be construed as an admission that the invention is not entitled to antedate such publications by virtue of prior invention.

Definitions

The term “peptidic compound”, as used herein, intends a compound comprising units which are linked to one another primarily, but not exclusively, by peptide bonds. The units typically comprise coded amino acid residues, non-coded amino acid residues, and/or peptidomimetics. The term “peptide” as used herein refers to any compound produced by amide formation between a carboxyl group of one amino acid and an amino group of another. The peptidic compounds may be polymers of: (a) naturally occurring, coded or non-coded, amino acid residues; (b) polymers of non-naturally occurring amino acid residues, e.g. N-substituted glycines, amino acid substitutes, etc.; or (c) polymers of both naturally occurring and non-naturally occurring amino acid residues/substitutes. The term includes synthetic peptides. In other words, the subject peptidic compounds may be peptides or peptoids. Peptoid compounds and methods for their preparation are described in WO 91/19735, the disclosure of which is herein incorporated by reference. Peptidic compounds of the invention are preferably characterized by (1) oral bioavailability, (2) 30 or less residues per molecule; (3) reducing phosphate levels in serum; (4) non-toxic to mammals in doses which are sufficiently high to be therapeutic, e.g., 1 to 1,000 mg per day per 70 kg man; (5) increasing incorporation of phosphorus into bone; and (6) comprising at least one moiety which is phosphorylated, or at least one moiety which is capable of being phosphorylated in vivo or in vitro, or a combination of such moieties. Amino acids are sometimes referred to herein by standard three-letter symbols (see, e.g., pages 58-59, “Biochemistry” Second Ed., Voet and Voet, eds. (1995) John Wiley & Sons, Inc.).

The terms “treatment”, “treating”, and “treat” are used herein to generally mean obtaining a desired pharmacologic and/or physiologic effect. The effect may be prophylactic in terms of completely or partially preventing a disease or symptom therof and/or may be therapeutic in terms of a partial or compete cure for a disease and/or adverse effect attributable to the disease. “Treatment” as used herein covers any treatment of a disease or condition in a mammal, particularly a human, and includes: preventing the disease or condition from occurring in a subject which may be predisposed to the disease or condition but has not yet been diagnosed as having it; inhibiting the disease or condition, i.e., arresting its development; relieving the disease, i.e., causing regression of, ameliorating, or palliating, the disease or condition. Treating includes preventing hyperphosphatemia from occurring, reducing phosphate levels in patients with hyperphosphatemia, and decreasing the ratio at which phosphate levels might rise in patients with hyperphosphatemia.

An “effective amount” or “therapeutic amount” is an amount sufficient to effect beneficial or desired clinical results. An effective amount can be administered in one or more administrations.

The term “osteoporosis” is intended to refer to any condition involving a reduction in the amount of bone mass or substance resulting from any cause, and in particular, from demineralization of the bone, postmenopausal or peri-menopausal estrogen decrease, disease or nerve damage.

The terms “subject”, “individual” and “patient” are used interchangeably herein to refer to any mammal, including a human. A variety of individuals are treatable according to the subject methods. Generally such individuals are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the subjects will be humans.

A “condition related to elevated phosphate levels in the serum” is one that is results from, directly or indirectly, abnormal levels of phosphate in body fluids (generally elevated levels), including, but not limited to, serum. It is also one which is an indicia of a condition related to (i.e., occurring as a direct or indirect consequence of) elevated phosphate levels in the serum. Such conditions include, but are not limited to, hyperphosphatemia, secondary hyperparathyroidism, renal osteodystrophy, soft tissue calcification, vascular calcification, osteoporosis, osteomalacia, bone loss, and/or bone weakness.

The term “bone loss” refers to any condition in which the bone mass, substance, or matrix or any components of the bone, such as calcium and/or phosphorus, is decreased or weakened. Individuals to be treated with serum phosphate level-reducing compounds of the invention include dialysis patients, especially those with serum phosphorus levels above about 6.0 mg/dl.

A “biological sample” encompasses a wide variety of sample types obtained from an individual for use in diagnostic or monitoring assays. The term encompasses blood, serum and other liquid samples of biological origin, and solid tissue samples such as a biopsy specimen. The term also includes samples that have been manipulated in any way after their procurement, such as be treatment with reagents, solubilization, or enrichment for certain components.

Overview of the Invention

The present invention provides compositions comprising compounds that are effective in reducing serum phosphate levels in an individual, and are therefore effective in treating conditions related to elevated phosphate levels in the serum. In some embodiments, these compounds comprise at least one moiety capable of being phosphorylated in vitro or in vivo. In other embodiments, these compounds comprise one or more phosphorylated moieties. In other embodiments, these compounds comprise at least one moiety capable of being phosphorylated in vitro or in vivo and at least one moiety which is phosphorylated. These differ from previously disclosed “phosphate binders” in that they are not simply ion-exchangers. They are further distinguished from previously disclosed phosphate binders in that they need not be administered in amounts stoichiometric with the amount of excess phosphate in the serum, but are effective at low doses. Surprisingly, such low doses of orally-administered synthetic peptidic compounds, including those that are fully phosphorylated, exhibited the ability to reduce serum phosphate levels in an animal disease model, as described herein. Further, not only the fully phosphorylated compounds, but also the compounds which contain moieties capable of being phosphorylated, demonstrated this ability. Furthermore, these peptidic compounds exhibited such activity even when administered without regard to mealtime.

Not only do these peptidic compounds lower serum phosphate at doses significantly lower and less frequent than those of phosphate binders, but also they increase bone phosphorus content and enhance bone strength without hypercalcemia or other adverse effects. Therefore, the present invention satisfies a pressing clinical need by demonstrating that this series of synthetic peptides and phosphopeptides are a safe, effective, and may be administered in an easily compliant method and result in controlling serum phosphate in hyperphosphatemic conditions.

Peptidic Compounds of the Invention

The present invention provides peptidic compounds, particularly synthetic peptidic compounds, which are useful in reducing serum phosphate levels in an individual. In some embodiments, peptidic compounds of the invention comprise at least one moiety that is capable of being phosphorylated, either synthetically in vitro, or by a physiological enzyme in vivo, such that a phosphate group is covalently bound to the moiety. In other embodiments, peptidic compounds of the invention comprise at least one moiety that is phosphorylated. In other embodiments, peptidic compounds of the invention comprise at least one moiety that is capable of being phosphorylated, either synthetically in vitro, or by a physiological enzyme in vivo, such that a phosphate group is covalently bound to the moiety; and at least one moiety that is phosphorylated.

The peptidic compounds of the present invention may be either linear, branched or cyclic peptides, and are comprised of monomer units consisting of a unit (I) selected from the group consisting of any naturally occurring amino acid and an amino acid residue of the general structural formula (I′): 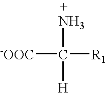
wherein R₁ is any moiety connectable (i.e., can be covalently linked) to the carbon atom; and
(b) an amino acid residue of the general structural formula (II): 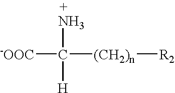
   and R₂ is any moiety which is connectable (i.e., can be covalently linked) to the carbon atom, and which is phosphorylated, or capable of being phosphorylated (i.e., a phosphate group covalently bound to the moiety), including, but is not limited to, amino acid side chains; sugars; nucleosides; nucleotides including nucleotide monophosphates and nucleotide diphosphates; sugar alcohols; compounds such as pyruvate, which, when phosphorylated give rise to enol phosphates; guanidines, such as arginine, and creatine; peptidomimetics which are phosphorylated or which are capable of being phosphorylated.

Amino acid side chains include those from any coded or non-coded amino acid which is phosphorylated or which is capable of being phosphorylated by a physiological enzyme, including, but not limited to, serine, threonine, tyrosine, histidine, arginine, and cysteine.

Sugars include, but are not limited to, ribose, glucose, inositol, and fructose. Nucleosides include, but are not limited to, adenine, guanine, cytosine, uracil, or thymine. Sugar alcohols include, but are not limited to, glycerol. Glycerol can further be esterified with a fatty acid.

Phosphorylation of R₂ can be carried out in vitro, chemically or enzymatically, or in vivo by a physiological enzyme present in the subject being treated. Enzymes which catalyze covalent linkage of a phosphate group to a moiety are phosphoryl transferases and include, e.g., kinases, and phosphorylases.

R₂ can be attached directly to the carbon atom, i.e., n=0. Alternatively, a short linker can be present between the carbon atom and R₂, e.g., n=1 to about 10.

In some embodiments, R₂ is selected from the group consisting of
wherein X is
H,

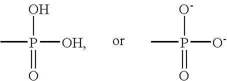

The amino acids contained in the polypeptide may be either the D- or L-isomer, with naturally occurring L-forms preferred.

In one embodiment, monomer unit (I) is a naturally-occurring amino acid and R₁ of monomer unit (I′) is defined such that (I′) is, alanine, cysteine, aspartic acid, glutamic acid, phenylalanine, glycine, histidine, isoleucine, lysine, leucine, methionine, glutamine, proline, arginine, serine, threonine, valine, tyrosine and tryptophan, asparagine, ornithine, valine, leucine, isoleucine, phenylalanine, threonine, tyrosine, aspartic acid, glutamic acid, and/or γ-carboxyl glutamic acid. More preferably, R₁ is defined such that (I′) is glycine, alanine or serine, and most preferably, glycine.

In another preferred embodiment, monomer unit (II) is serine, threonine, tyrosine, phosphoserine, phosphothreonine or phosphotyrosine, and most preferably serine orphosphoserine.

In some embodiments, monomer unit (I) is glycine, and monomer unit (II) is serine. In some of these embodiments, a peptidic compound comprises 7 (Ser-Gly) units.

In another embodiment, the number of monomer units contained in the polypeptide consists of at least 2 and less than 30 units, and more preferably 4 to 14 units.

In another embodiment, monomeric unit (II) is phosphorylated, and comprises about 3%, generally about 5%, generally at least about 10%, usually at least about 15%, more usually at least about 20%, more preferably at least about 25% or more, and up to about 50% of the peptidic compound.

When administered in an effective amount to an individual, the compounds of the invention are effective in reducing phosphate levels, as generally indicated by serum phosphate levels, in the individual, i.e., a compound of the invention is effective in reducing a phosphate level at least about 5%, generally at least about 10%, usually at least about 15%, more usually at least about 20%, more preferably at least about 25% or more, compared to a level before treatment. A peptidic compound of the invention may also result in a degree of reduction in serum phosphate levels such that normal physiological serum phosphate levels are attained. Thus, a “therapeutically effective amount”, or an “effective amount” of a peptidic compound of the invention is one that, when administered in a composition to an individual, results in a reduction in a phosphate level of at least about 5%, generally at least about 10%, usually at least about 15%, more usually at least about 20%, more preferably at least about 25% or more, compared to a level before administering the peptidic compound, or which results in a degree of reduction in serum phosphate levels such that normal physiological serum phosphate levels are attained.

Methods for measuring phosphate levels in an individual are known in the art and can be used to assess whether a given compound is effective in reducing a phosphate level in an individual. For example, the biological sample is burned to remove carbon, then ashed in a 600° C. oven. Concentrated HCl is added to the sample to dissolve the phosphorus. The phosphorus is then determined with a ferrous sulfate-ammonium molybdate reagent. Intensity of blue color is determined at 700 nm with a spectrophotometer. Alternatively, after ashing the sample, phosphorus content can be measured by atomic absorptiometry. Biological sample which can be tested for measuring phosphate level in an individual include, but are not limited to, serum, plasma, blood, and tissue samples. Typically, phosphate levels will be measured in serum.

In some embodiments, a peptidic compound of the invention is also effective in increasing bone phosphorus content, i.e., increasing incorporation of phosphorus into bone, when administered in an effective amount to an individual. Thus, in these embodiments, a compound of the invention, in addition to reducing serum phosphate levels in an individual, increases bone phosphorus content by at least about 1%, generally at least about 2%, typically at least about 3%, more preferably at least about 4% or more, when compared to a level before administering the peptidic compound. Thus, in these embodiments, a “therapeutically effective amount”, or an “effective amount” of a compound of the invention is one that, when administered in a composition to an individual, results in an increase in bone phosphorus content by at least about 1%, generally at least about 2%, typically at least about 3%, more preferably at least about 4% or more, when compared to a level before administering the peptidic compound.

In some embodiments, a peptidic compound of the invention, when administered in an effective amount to an individual, results in both an increase in bone phosphorus content by at least about 1%, generally at least about 2%, typically at least about 3%, more preferably at least about 4% or more, when compared to a level before administering the peptidic compound, and in a reduction in a serum phosphate level of at least about 5%, generally at least about 10%, usually at least about 15%, more usually at least about 20%, more preferably at least about 25% or more, compared to a level before administering the peptidic compound, or to any degree in which normal physiological levels of serum phosphate are attained.

Methods for measuring bone phosphorus content are known in the art and can be used to assess whether a given compound is effective in increasing bone phosphorus content in an individual. As an example, bone can be ashed, and the phosphorus content measured by atomic absorbtiometry.

Furthermore, indirect indications of increased bone phosphorus content, such as increased bone strength, can be measured to assess whether a given compound is effective in increasing bone phosphorus content in an individual. In some embodiments, peptidic compounds of the invention, when administered in an effective amount to an individual, can also increase bone strength in the individual. Thus, in some embodiments, an effective amount of a peptidic compound of the invention is one that results in an increase of at least about 5%, generally at least about 10%, more preferably at least about 15% or more, in bone strength, when compared to bone strength before administering the peptidic compound. Any known method for measuring bone strength can be used, including, but not limited to, those described in the Examples. For example, a three-point bending analysis can be performed to assess mechanical bone strength, as described in the Examples. Non-invasive in vivo measurements of bone strength and bone density are also known in the art, and can be used to assess whether a peptidic compound is effective in increasing bone strength and/or increasing bone density, as an indirect indication of an increase in bone phosphorus content. These methods include, but are not limited to, dual-energy x-ray absorptiometry (DEXA); ultrasound measurements of bone density (see, e.g., U.S. Pat. No. 5,879,301); vibrational analysis to measure bending stiffness of bones (see, e.g., U.S. Pat. No. 5,368,044); peripheral quantitative computed tomography (pQCT), as described, for example, in Schiessl et al. (1996) “New developments in diagnostics and therapy”, in Pediatric Osteology, E. Schoenau, ed. Elsevier Science; and methods such as those described in U.S. Pat. Nos. 5,931,795 and 5,778,045.

Method of Production

The peptidic compounds of the invention may be prepared by common peptide synthesis, and other standard organic chemistry synthesis methodologies, generally available in the art. For example, the following method may be used.

For the synthesis of fully phosphorylated peptides, the protected dipeptide required for the synthesis of repeated peptides can be prepared by a solution phase method. Preparations of protected peptides resins are obtained by the DCC-HOBt-mediated coupling of the protected dipeptide on H-Ser(OPO3Me2)-Gly-Merrifield resin which is synthesized by coupling of Boc-Ser(OPO3Me2)—OH on H-Gly-Merrifield resin followed by trifluoroacetic acid (TFA)-mediated Boc deprotection. Crude deprotected peptides are produced by treating the completed protected peptide resins (Boc-(Ser(OPO3Me2)-Gly)n-Merrifield resin, n>1, preferably, n=2-7) with a two-step deprotecting procedure consisting of high acidic (IM TMSOTf-thioanisole in TFA, m-cresol, EDT)-and low acidic (1 M TMSOTf-thioanisole in TFA, m-cresol, EDT+additives (TMSOTf+DMS)). Pure peptides are obtained by HPLC purification of crude peptides. The synthesized peptides are then characterized by ion-spray mass spectrometry.

For the synthesis of non-phosphorylated or partially phosphorylated peptides, protected Ser-containing peptide unit (Boc-Ser(Bzl)-Gly-OH) can be used to incorporate one or more non-phosphorylated Ser residue(s). The dipeptide unit can be introduced into a protected peptide resin in a manner similar to that employed for the synthesis of fully phosphorylated peptide. Partially phosphorylated protected peptide resin can be subjected to a two-step deprotection procedure consisting of high acidic (first step: 1 M TMSO Tf-thioanisole in TFA, m-cresol, ethanedithiol) and low acidic (second step: first step plus TMSO Tf/dimethylsulfide) steps, which yields deprotected partly phosphorylated peptides. Treatment of protected non-phosphorylated peptide resin with 1 M TMSO Tf-thioanisole in TFA, m-cresol, ethanediol, yields the deprotected non-phosphorylated peptide.

The above-described procedures relate to methods of synthesizing Ser-Gly dipeptides (phosphorylated and non-phosphorylated). As will be apparent to those skilled in the art, similar procedures can be used to incorporate other phosphorylated and/or non-phosphorylated residues. For example, H-Thr(OPO₃Me₂)-Gly-Merrifield resin and/or (Boc-Thr(Bzl)-Gly-OH can be used in a similar manner to synthesize phosphorylated threonine- and non-phosphorylated threonine (Thr)-containing molecules.

For preparation of longer peptidic compounds containing more than fourteen amino acids, including phosphorylated amino acids, a combination of recombinant DNA methodologies and enzymatic or organic synthesis methods may be more suitable.

For example, the peptidic compound may be produced by first culturing a cell line transformed with a polynucleotide sequence which encodes the amino acid sequence of the basic polypeptide. After producing such a polypeptide by cell culture, the hydroxyl groups of the appropriate amino acid can be substituted by phosphate groups using organic synthesis or enzymatic methods with phosphorylation enzymes such a phosphorylase, or a kinase. In the case of serine-specific phosphorylation, more specific enzymes such as serine kinases may be used.

Formulations

The peptidic compounds of the invention are formulated for administration in a manner customary for administration of such materials. Accordingly, the present invention provides compositions comprising a peptidic compound of the invention and a pharmaceutically acceptable excipient. These compositions (also referred to herein as “formulations”), which comprise an effective amount of a peptidic compound of the invention and a pharmaceutically acceptable excipient, are suitable for administration to individuals in unit dosage forms, sterile parenteral solutions or suspensions, oil in water or water in oil emulsions, and the like. Typical formulations and pharmaceutically acceptable excipients are those provided in Remington's Pharmaceutical Sciences, latest edition, Mack Publishing Company, Easton, Pa. The percentage of active ingredient (i.e., peptidic compound of the invention) in such formulations will be 0.1% to 99% and the percentage of carrier will be 1.0 to 99.9%. The wide range of formulation possibilities are provided in part due to the high degree of solubility of compounds of the type described above. Preferably, the peptidic compounds are administered orally or by injection, including intramuscular, intravenous, subcutaneous or peritoneal injection routes. However, other modes of administration may also be used provided means are available to permit the compounds to enter the systemic circulation, such as transmucosal or transdermal formulations, which can be applied as suppositories, skin patches, intranasally, via inhalation, via nebulization, or trans-rectal route. In addition, local administration such as by cerebrospinal injection or injection directly into bone or fracture sites may also be used. Any suitable formulation which effects the transfer of the compound to the bloodstream or locally to the bone may properly be used.

For injection, suitable formulations generally comprise aqueous solutions or suspensions using physiological saline, Hank's Solution, or other buffers optionally including stabilizing agents or other minor components. Liposomal preparations and other forms of microemulsions can also be used. The compounds may also be supplied in lyophilized form and reconstituted for administration. Transmucosal and transdermal formulations generally include agents which facilitate transition of the mucosal or dermal barrier, such as bile salts, fusidic acid and its analogs, various detergents, and the like.

For oral administration suitable vehicles are tablets, dragees or capsules having talc and/or a carbohydrate carrier binder or the like, the carrier preferably being lactose and/or corn starch and/or potato starch. A syrup, elixir or the like can be used wherein a sweetened vehicle is employed. Sustained release compositions can be formulated including those wherein the active component is protected with differentially degradable coatings, e.g., by microencapsulation, multiple coatings, etc.

The peptidic compounds of the invention are generally highly water soluble and thus, are easily formulated as an aqueous solution for oral, parenteral or mucosal administration. Solubility increases with the number of phosphorylated residues in a single polypeptide molecule. Water solubility and stability in aqueous solution is often one of the major problems associated with the administration of many peptide drugs. The polypeptides of the invention provide a considerable advantage in this respect.

The nature of the formulation will depend to some extent on the nature of the compound chosen and a suitable formulation is prepared using known techniques and principles of formulation well known to those in the art.

Methods Using Peptidic Compounds of the Invention

The present invention provides methods using the peptidic compounds of the invention, including methods for reducing serum phosphate levels in an individual; methods for increasing incorporation of phosphorus into bone in an individual; and methods of increasing bone strength in an individual. The methods generally comprise administering an effective amount of a peptidic compound of the invention, whereby a therapeutic effect is achieved. “Effective amounts” of peptidic compounds of the invention are as described above.

In one embodiment, the invention provides a method for a reducing serum phosphate level in an individual, comprising administering an effective amount of a peptidic compound of the invention to the individual. Typically, the peptidic compound is administered as a composition with a pharmaceutically acceptable excipient(s). These methods are useful to treat a variety of conditions, including hyperphosphatemia. Accordingly, the invention further provides a method for treating hyperphosphatemia in an individual, comprising administering an effective amount of a peptidic compound of the invention, usually in a pharmaceutical composition.

In another embodiment, the invention provides a method for increasing bone phosphorus content in an individual, comprising administering an effective amount of a peptidic compound of the invention to the individual, generally in a formulation with pharmaceutically acceptable excipient(s). In a further embodiment, the invention provides a method for increasing bone strength in an individual, comprising administering an effective amount of a peptidic compound of the invention to the individual, generally in a formulation with pharmaceutically acceptable excipient(s).

These methods are useful to treat a variety of diseases characterized by reduced bone phosphorus content. Accordingly, in a further embodiment, the invention provides a method for treating a bone disease in an individual, wherein the bone disease is characterized by reduced bone phosphorus content, comprising administering an effective amount of a peptidic compound of the invention to the individual, generally in a formulation with pharmaceutically acceptable excipient(s). Treating a bone disease, characterized by reduced bone phosphorus content, by administering a peptidic compound of the invention results in increased bone phosphorus content and increased bone strength, compared to bone phosphorus content and bone strength in the individual before treatment.

Peptidic compounds are generally administered in formulations (pharmaceutical composition), as described above. Effective amounts are those described above. The appropriate dosage level will also vary depending on a number of factors including the nature of the subject to be treated (age, sex, weight, etc.), the particular nature of the condition to be treated and its severity, the particular compound used as active ingredient, the mode of administration, the formulation, and the judgment of the practitioner. Generally, dosages will be in the range of 100 μg/kg to 5 mg/kg, preferably 10 mg/kg to 20 mg/kg at a single dosage. Repeated administration may be required according to protocols to be determined considering the variables set forth above. For example, the formulations can be repeatedly administered once a day or more over a period of 30 days or more Typically, daily administration over a period of limited period of days may be required or administration by intravenous means may be continuous. For chronic conditions, administration may be continued for longer periods, e.g., months or years, as necessary.

Subjects who would benefit from administration of the peptidic compounds of the invention are those who, for any reason, have elevated levels of serum phosphate, for example, those with serum phosphate levels greater than about 6.0 mg/dl and/or those who have a disorder characterized by reduced bone phosphorus content and/or bone weakness. Subjects may further exhibit bone loss or weakening. Particular, conditions which may be especially amenable to treatment include, but are not limited to, renal insufficiency, hyperparathyroidism, pseudohyperparathyroidism, overmedication with phosphate salts, hyperphosphatemia, as well as conditions related to any of the foregoing, including, but not limited to, osteoporosis, renal osteodystrophy, osteomalacia, osteodystrophy resulting from other causes, Paget's Disease or osteolysis mediated by cancer, and fractures. Subjects are preferably human, but may include any mammal.

Whether a therapeutic effect has been achieved can be determined by methods known to those skilled in the art, and as described herein. Thus, for methods of reducing a serum phosphate level in an individual and methods of treating hyperphosphatemia, comprising administering an effective amount of a peptidic compound of the invention, serum phosphate levels can be monitored. Similarly, bone phosphorus content or bone strength can be measured using standard methods, including non-invasive methods such as those described above.

The following examples are put forth so as to provide those of ordinary skill in the art with a complete disclosure and description of how to make and use the present invention, and are not intended to limit the scope of what the inventors regard as their invention, nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Celcius, and pressure is at or near atmospheric.

### EXAMPLES

### Example 1

Prevention of Hyperphosphatemia and Bone Weakening in an Osteoporosis Model

Ovariectomized rats were chosen for the efficacy study of a series of peptides. After a six (6) day quarantine/acclimation period, forty (40) ovariectomized female Sprague Dawley outbred (Crl:CD®IGS BR) rats were randomly allocated into five experimental groups of eight animals each. A sixth group was composed of eight sham-operated Sprague Dawley rats. The experimental design consisted of four test-article groups and two vehicle control groups (one ovariectomized and one sham-operated). Each rat received a single daily oral dose for 84 days of either 0.9% Sodium Chloride for Injection (controls) or 200-208 μg/kg of peptide as specified in Table 1. Doses were based upon a target dose of 50 μg/animal/day and group mean body weights determined at study initiation (Day 0). “Sham” indicates sham-operated rats; and “OVX” indicates ovariectomized rats.

TABLE 1

| Group | n | Test System | Treatment | Dose | Route/Duration |
|---|---|---|---|---|---|
| 1 | 8 | OVX | (Ser-Gly)₇ | 200 | Oral; daily |
| | | | | | for 84 days |
| 2 | 8 | OVX | (Ser-Gly)₅ | 207 | Oral; daily |
| | | | (Pse-Gly)₂ | | for 84 days |
| 3 | 8 | OVX | (Ser-Gly)₂ | 208 | Oral; daily |
| | | | (Pse-Gly)₅ | | for 84 days |
| 4 | 8 | OVX | (Pse-Gly)₇ | 205 | Oral; daily |
| | | | | | for 84 days |
| 5 | 8 | OVX | 0.9% Sodium | 0 | Oral; daily |
| | | | Chloride | | for 84 days |
| 6 | 8 | Sham | 0.9% Sodium | 0 | Oral; daily |
| | | | Chloride | | for 84 days |

The peptides used were as follows:

| | |
|---|---|
| (Ser-Gly)7=Linear peptide of: | |
| Ser-Gly-Ser-Gly-Ser-Gly-Ser-Gly-Ser-Gly-Ser-Gly-Ser-Gly | (SEQ ID NO:1) |
| where Pse= O-phosphoserine; | |
| (Ser-Gly)₅(Pse-Gly)₂=Linear peptide of: | |
| Ser-Gly-Ser-Gly-Ser-Gly-Ser-Gly-Ser-Gly-Pse-Gly-Pse-Gly; | (SEQ ID NO:2) |
| (Ser-Gly)₂(Pse-Gly)₅=Linear peptide of: | |
| Ser-Gly-Ser-Gly-Pse-Gly-Pse-Gly-Pse-Gly-Pse-Gly-Pse-Gly; | (SEQ ID NO:3) |
| and | |
| (Pse-Gly)7=Linear peptide of: | |
| Pse-Gly-Pse-Gly-Pse-Gly-Pse-Gly-Pse-Gly-Pse-Gly-Pse-Gly. | (SEQ ID NO:4) |

The dose range of 200-208 μg/kg/day employed in the study represents the target dose (50 μg/animal/day) in animals weighing 224-264 g at study initiation. The test samples for groups 1, 2, 3, and 4 were dissolved in 0.9% Sodium Chloride for injection and administered at once. All dosing was accomplished by oral gavage (dose volume 1.0-1.5 mL, adjusted weekly for bodyweight gain). Certified Rodent Diet #5002 (Purina Mills, Inc., St. Louis, Mo.) was provided during the quarantine/acclimation period and throughout the study.

Clinical signs were observed once daily throughout the quarantine/acclimation and treatment periods. Individual body weights were recorded at the initiation of the dosing, once weekly throughout the treatment phase, and at necropsy. Animals were sacrificed approximately 24 hours after the administration of the final dose (Day 84). Terminal whole blood samples were collected from each animal for serum chemistry (i.e. measurement of serum calcium and phosphorus) evaluations via terminal cardiocentesis. Femurs were submitted for post-mortem bone mechanical strength and composition analysis.

The left femur of each animal was reserved for bone composition analysis. First, the femur was dried until all moisture was eliminated. The femur was then ashed and the phosphorus content in the ash was quantitated by atomic absorptiometry.

The right femur of each animal was reserved for bone mechanical strength evaluation (i.e. three-point bending analysis). Femurs were placed on an apparatus in which two isolated points of the long bone were supported. Weight load was applied at the center of two supporting points toward the direction in which the bone is bent. Femur supports had a diameter of 1 mm with a lower span of 20 mm. Femurs were positioned with the anterior side toward the center load, and the posterior side toward the two supports. The loading was done in an Instron Model #1122 materials testing machine (Canton, Mass.). Testing was conducted with a displacement of 2.0 mm/minute. Load and displacement were recorded at 0.002 mm displacement intervals. Experiments were controlled using a computer-based data acquisition system running ASYST Scientific Software (Keithley-Metrabyte; Taunton, Mass.). The following data were generated: Maximum Load (in Newton, N); Initial Stiffness (in N/mm); Energy at Maximum (in Nmm)

The value of the load was plotted against the deflection generating the load-deflection curve. Two points in the initial linear portion of the load-deflection curve were chosen by the operator at the time of testing. A line was constructed through the chosen points, the slope of which is the initial stiffness. The maximum load is the largest value of the load recorded during the flexural test. The energy at maximum is the area under the load-deflection curve up to the maximum load point.

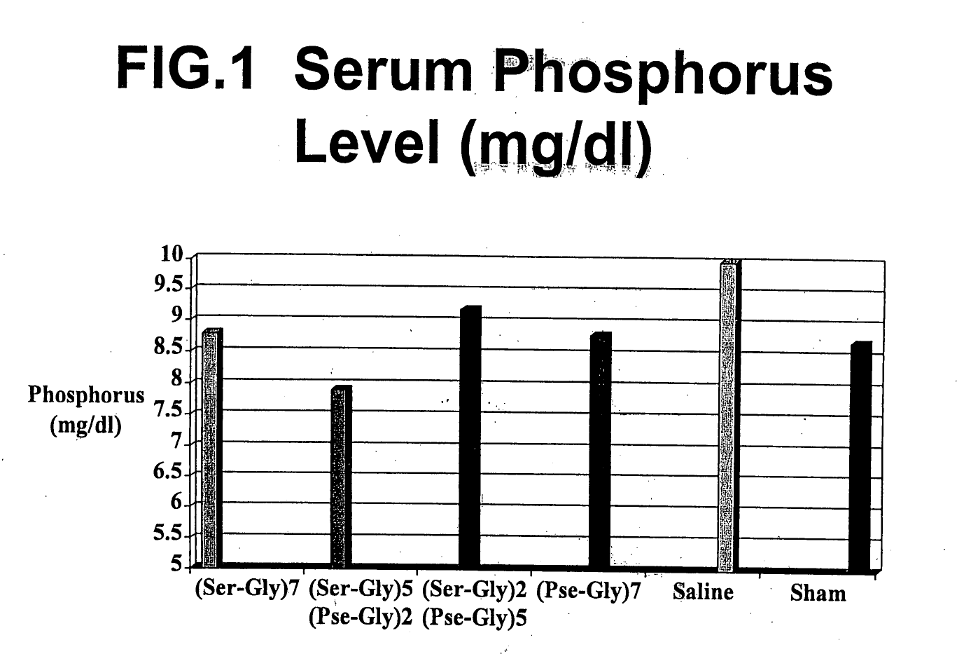

As shown in FIG. 1, serum phosphorus concentration was higher in ovariectomized rats than in sham operated controls at the end of the study period (Group 5 vs. Group 6). The serum phosphorus concentrations of all treated groups (Groups 1-4) were lower than those of the saline-treated ovariectomized controls, and in the Group 2 rats treated with (Ser-Gly)₅(Pse-Gly)₂ (SEQ ID NO:2), were equivalent to or even lower than sham-operated controls.

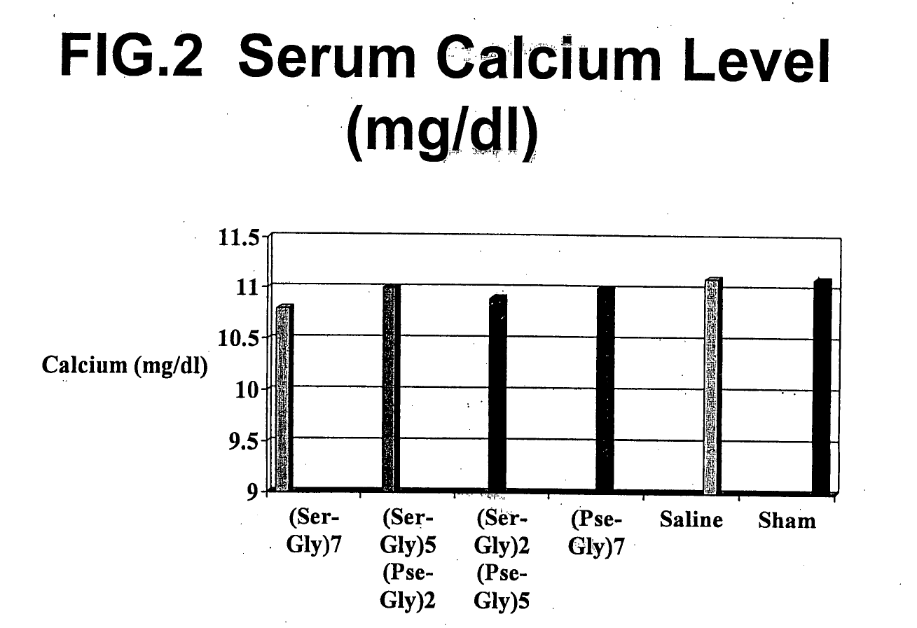

FIG. 2 demonstrates the serum calcium concentration for each of the treatment and control groups. Serum calcium levels appeared unchanged by ovariectomy (Group 5 vs. Group 6) and were similar among peptide-treated and saline-treated ovariectomized animals (Groups 1-4 vs. Group 5).

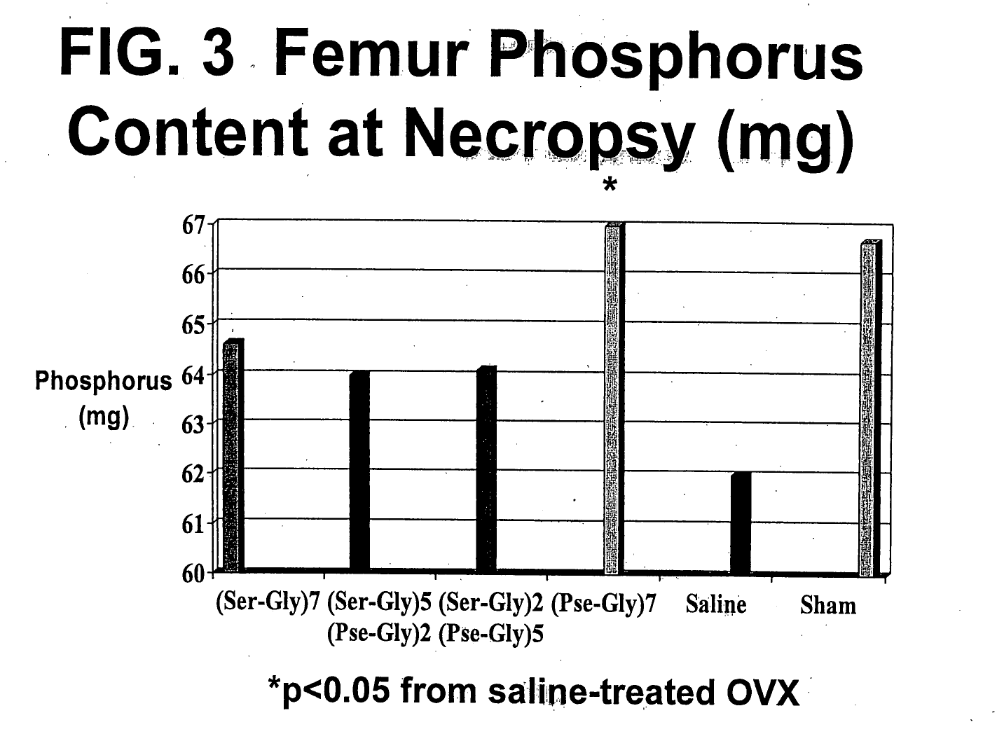

FIG. 3 displays the phosphorus content in the femurs for each of the treatment and control groups. Ovariectomy resulted in a decrease in bone phosphorus content (Group 5 vs. Group 6). Each of the treatment groups had increased bone phosphorus content relative to the ovariectomized controls (Groups 1-4 vs. Group 5). In the case of the Group 4 rats, (Pse-Gly)₇ SEQ ID NO:4, there was a statistically significant increase in bone phosphorus content as compared to the saline-treated ovariectomized rats and a restoration of bone phosphorus levels to those of the sham-operated controls.

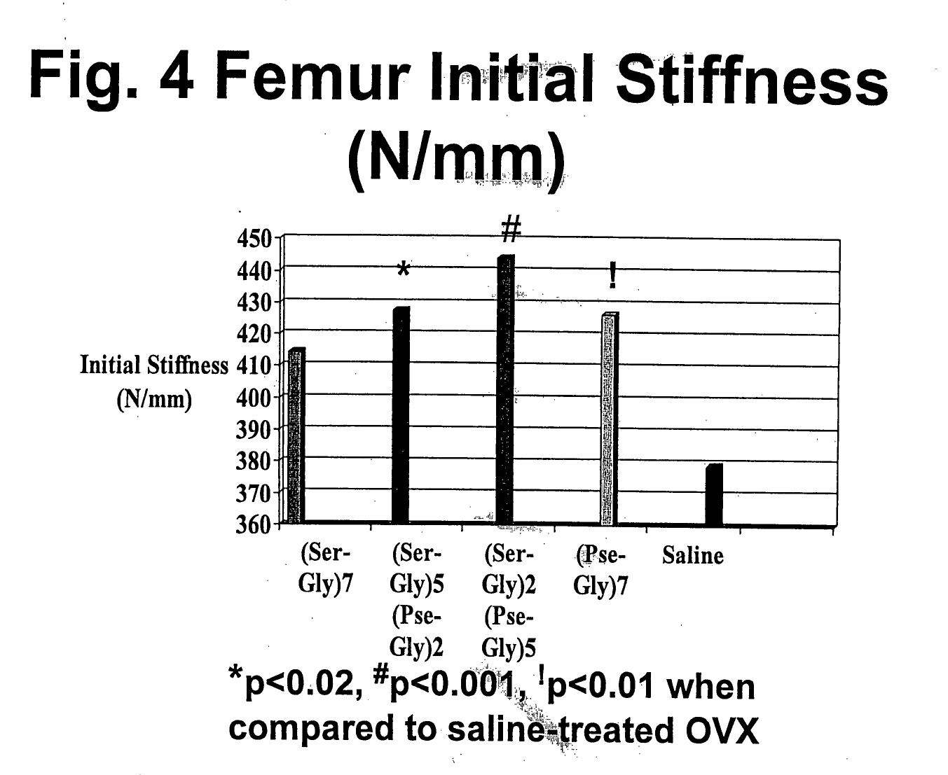

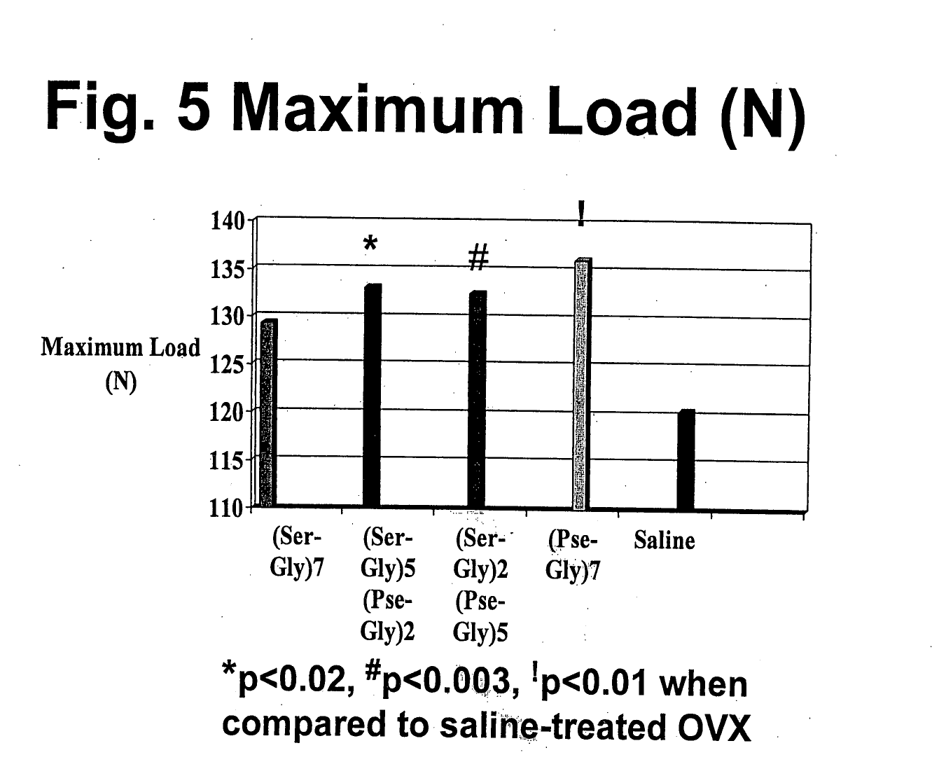

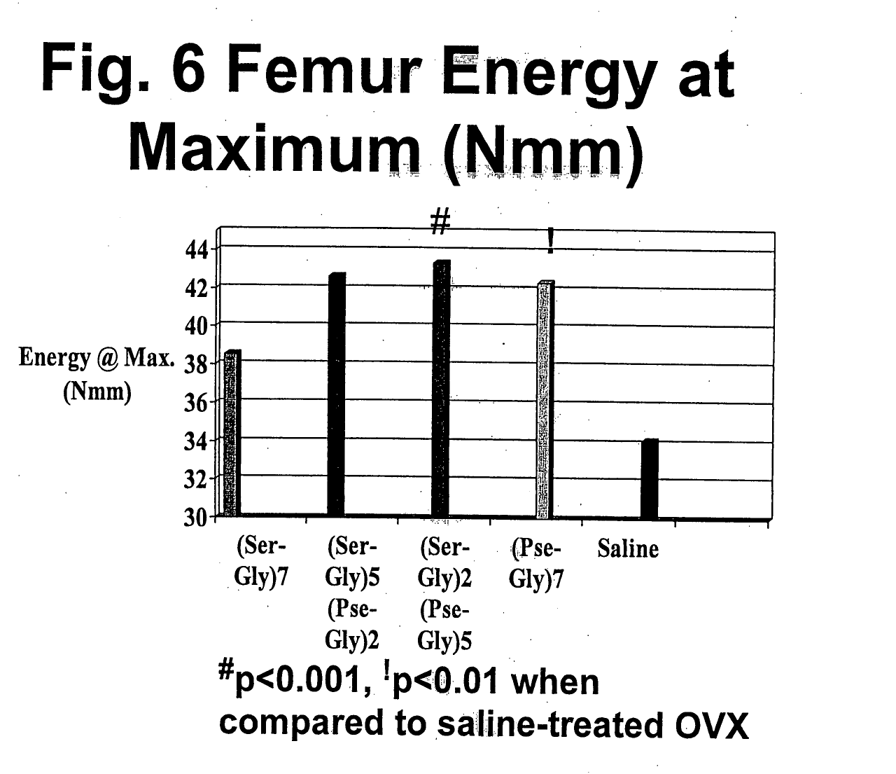

FIGS. 4, 5, and 6 show the initial bone stiffness, maximum load, and energy at maximum, respectively. As exhibited in these figures, each of the treated groups demonstrate an increase in each of these mechanical strength parameters as compared to the saline-treated ovariectomized controls. A statistically significant increase in each of these parameters was observed in the (Ser-Gly)₅(Pse-Gly)₂ (SEQ ID NO:2); (Ser-Gly)₂(Pse-Gly)₅ (SEQ ID NO:3); and (Pse-Gly)₇ (SEQ ID NO:4) groups respectively (Groups 2, 3, and 4 vs. Group 5).

Throughout the 84 day experimental period, all animals in the study appeared completely healthy. Furthermore, no complications, toxicity, or adverse side effects were observed.

The results for serum alkaline phosphatase levels, serum Ca²⁺ levels, serum inorganic phosphate levels, and urine Ca²⁺ levels are summarized in Table 2, below. Sham=sham operated rats; OVX=ovariectomized rats. (Ser-Gly)₇ is a peptidic compound having the sequence given as SEQ ID NO: 1. (Ser-Gly)₅ (Pse-Gly)₂, (Ser-Gly)₂ (Pse-Gly)₅, and (Pse-Gly)₇ are peptidic compounds having the sequence (SerGly)₇, wherein, on average, 2, 5, and 7, respectively, of the seven serines are phosphorylated.

TABLE 2
Serum Alkaline Phosphatase (AP), Serum Ca²⁺, Serum Inorganic
Phosphate, and Urine Ca²⁺ Levels

| | Serum AP | Serum Ca²⁺ | Serum Pᵢ | Urine Ca²⁺ |
|---|---|---|---|---|
| | (U/L) | (mg/dl) | (mg/dl) | (mg/dl) |
| Sham | 64 ± 8 | 11.1 ± 0.8 | 8.7 ± 1.2 | 10.1 ± 9.4 |
| OVX Control | 105 ± 30 | 11.1 ± 0.5 | 10.0 ± 2.1 | 31.6 ± 19.3 |
| (Ser-Gly)₇ | 134 ± 53 | 10.8 ± 0.6 | 8.8 ± 1.0 | 26.7 ± 22.9 |
| (Ser-Gly)₅ | 118 ± 18 | 11.0 ± 0.3 | 7.9 ± 0.9 | 26.6 ± 14.1 |
| (Pse-Gly)₂ | | | | |
| (Ser-Gly)₂ | 121 ± 30 | 10.9 ± 0.6 | 9.2 ± 1.3 | 29.5 ± 20.0 |
| (Pse-Gly)₅ | | | | |
| (Pse-Gly)₇ | 125 ± 43 | 11.0 ± 0.5 | 8.8 ± 1.3 | 20.0 ± 12.5 |

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1-19. (Canceled)

20. A method of reducing serum phosphate levels in a human patient by more than 5% by:
administering to the patient a peptidic compound comprising monomer units selected from:
(a) a unit (I) selected from the group consisting of a coded amino acid, a non-coded amino acid, and a synthetic amino acid, of the general structural formula (I′): 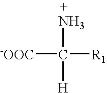
wherein R₁ is any moiety connectable to the carbon atom; and
(b) a unit (II) selected from the group consisting of a coded amino acid, a non-coded amino acid, and a synthetic amino acid, of the general structural formula: 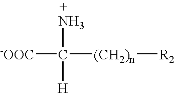
wherein R₂ is any moiety which is phosphorylated or which is capable of being phosphorylated, wherein n=0 to 10.

21. The method of treating hyperphosphatemia, comprising:
administering to an individual a therapeutically effective amount of a composition comprising a pharmaceutically acceptable excipient and a peptidic compound characterized by (a) oral bioavailability, (b) 4 to 30 amino acid residues, and (c) having at least one amino acid residue which is phosphorylated or which is phosphorylatable in vivo or in vitro.

22. The method of claim 21, wherein the composition comprises 1 to 1,000 mg of the peptidic compound.

23. The method of claim 21, wherein the individual is a mammal.

24. The method of claim 23, wherein the peptidic compound is further characterized by reducing serum phosphate levels 5% or more in the mammal.

25. The method of claim 21, further comprising:
repeatedly administering the composition once a day or more over a period of 30 days or more.

26. The method of claim 21, wherein said peptidic compound comprises monomer units selected from:
(a) a unit (I) selected from the group consisting of a coded amino acid, a non-coded amino acid, and a synthetic amino acid, of the general structural formula (I′): 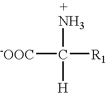
wherein R₁ is any moiety connectable to the carbon atom; and
(b) a unit (II) selected from the group consisting of a coded amino acid, a non-coded amino acid, and a synthetic amino acid, of the general structural formula: 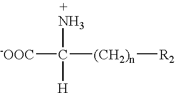
wherein R₂ is any moiety which is phosphorylated or which is capable of being phosphorylated, wherein n=0 to 10.

27. The method of claim 26, wherein R₁ is —H.

28. The method of claim 26, wherein R₂ of each monomer unit is independently selected from the group consisting of —CH₂OX, —CH(OX)—CH₃, —CH₂(phenyl)-OX, wherein X is H, [chemical structure]
